(19) United States
(12) Patent Application Publication
(10) Pub. No.: US 2019/0127295 A1
(43) Pub. Date: May 2, 2019

(54) Scalable And Robust Burner/Combustor And Reactor Configuration

(71) Applicant: SABIC Global Technologies B.V., Bergen op Zoom (NL)

(72) Inventors: Sreekanth Pannala, Sugar Land, TX (US); Balamurali Krishna Ramachandran Nair, Sugar Land, TX (US); Pankaj Singh Gautam, Evansville, IN (US); Krishnan Sankaranarayanan, Missouri City, TX (US); David West, Bellaire, TX (US); Istvan Lengyel, Lake Jackson, TX (US); Chunliang Wu, Katy, TX (US)

(21) Appl. No.: 16/302,699

(22) PCT Filed: May 9, 2017
(86) PCT No.: PCT/US2017/031770

§ 371 Date: Nov. 19, 2018

Related U.S. Application Data
(60) Provisional application No. 62/341,819, filed on May 26, 2016.

(51) Int. Cl. C07C 2/78; B01J 19/24; C07C 11/04; C07C 11/24; F23C 5/08; F23C 6/02; F23C 6/04

(52) U.S. Cl. CPC C07C 2/78; B01J 19/2485; C07C 11/04; C07C 11/24; F23C 2201/301; F23C 6/02; F23C 6/047; B01J 2208/00504; B01J 2219/00157; F23C 5/10

(57) ABSTRACT

Disclosed herein are processes, apparatuses, and systems for producing chemicals. One system may comprise a wall defining a chamber; a plurality of burners configured in an arrangement within the chamber, wherein each of the burners is supplied with a material and facilitates combustion of the material, and wherein the arrangement defines an inner volume disposed radially inwardly relative thereto; and an injector disposed within the inner volume and configured to introduce a feedstock into the chamber, wherein the plurality of burners provide thermal energy to facilitate thermal pyrolysis of the feedstock.

### FIELD OF DISCLOSURE

The present disclosure relates to thermal pyrolysis for mass production of chemicals. More specifically, the disclosure relates to scalable combustors and reactor configuration for controlled thermal pyrolysis of hydrocarbons from natural gas for mass production of chemicals.

### BACKGROUND

The use of thermal pyrolysis in the production of acetylene/ethylene has been extensively researched. As an example, such thermal conversion may be accomplished using: a) co-pyrolysis along with combustion; b) staged combustion followed by pyrolysis; and c) combustion phase followed by utilizing shock-waves to manipulate pyrolysis conditions.

A partial oxidation process developed by BASF Company represents an example one-step acetylene production process, in which natural gas substantially comprising methane serves for the hydrocarbon feed and pure oxygen as the oxidant. The general reactor configuration and mechanical design for this example single step partial process is described in U.S. Pat. No. 5,789,644. As a whole, the partial oxidation reactor system includes three major parts: the first part (top) is a mixing zone with a special diffuser, the second part (underneath) is a water-jacketed burner immediately followed by a reaction zone, and the third part is a quenching zone using water or heavy oil as a coolant. Aside from the description of the general process scheme, examples of certain feed ratios, especially the carbon-to-oxygen ratios are specified in U.S. Pat. No. 5,824,834.

As an additional example, acetylene can also be produced through a two-stage high temperature pyrolysis (HTP) process, for example, as described by Hoechst (GB 921,305 and 958,046). This process may include two main reaction zones followed by a quenching zone. The first reaction zone may serve as a stoichiometric combustor to supply the necessary endothermic heat of hydrocarbon pyrolysis taking place in the second reaction zone, into which a fresh hydrocarbon feed such as methane is introduced. In the quenching zone, water or heavy oil may be used as a coolant to the hot product gas from the pyrolysis zone. Similarly, a certain quantity of carbon will be formed in this two-step pyrolysis process. The amount of acetylene produced can also be increased by injection of methanol into the reaction zone during thermal cracking of hydrocarbons between 1000° C. and 1200° C., as described by Mitsubishi in U.S. Pat. No. 4,725,349.

The acetylene thus obtained can be used to make a variety of useful products via different synthesis routes. Notably, the acetylene can be converted to ethylene through a catalytic hydrogenation step. The process for hydrogenation of acetylene to ethylene in the presence of palladium-aluminum oxide (Pd/Al₂O₃) catalyst is also well known (U.S. Pat. No. 5,847,250).

In each of the thermal conversion processes listed above, one may attempt to create a narrow temperature range for methane exposure to maximize the yield of acetylene and ethylene. In addition, it may also be important to reduce the overall energy losses to ensure high efficiencies on a total fuel basis. However, the conventional processes and reactor configuration used in implementing the processes are plagued with materials issues as the temperatures are upward of 2000° C.

These and other shortcomings of the prior art are addressed by aspects of the present disclosure.

### SUMMARY OF THE DISCLOSURE

As described in more detail herein, the present disclosure provides processes, apparatuses, and systems for thermal pyrolysis in the mass production of chemicals.

In an aspect, a system may comprise: a wall defining a chamber; a plurality of burners configured in an arrangement within the chamber, wherein each of the burners is supplied with a material and facilitates combustion of the material, and wherein the arrangement defines an inner volume disposed radially inwardly relative thereto; and an injector disposed within the inner volume and configured to introduce a feedstock into the chamber, wherein the plurality of burners provide thermal energy to facilitate thermal pyrolysis of the feedstock.

In an aspect, a system may comprise: a wall defining a chamber; and a plurality of pyrolysis cells arranged within the chamber, wherein each pyrolysis cell comprises: a plurality of burners configured in an arrangement within the pyrolysis cell, wherein each of the burners is supplied with a material and facilitates combustion of the material, and wherein the arrangement defines an inner volume disposed radially inwardly relative thereto; an injector disposed within the inner volume and configured to introduce a feedstock into the pyrolysis cell, wherein the plurality of burners provide thermal energy to facilitate thermal pyrolysis of the feedstock.

In an aspect, a method of processing a hydrocarbon stream, the method comprising: causing combustion of a fuel to generate heat within a chamber via a plurality of burners configured in an arrangement within the chamber, wherein the arrangement defines an inner volume disposed radially inwardly relative thereto; and causing a feedstock to be introduced in the chamber via an injector disposed within the inner volume, wherein the heat generated by the combustion of the fuel facilitates thermal pyrolysis of the feedstock.

Controlling the pyrolysis zone temperature in a lower range would increase the yield of ethylene with respect to acetylene

### BRIEF DESCRIPTION OF THE FIGURES

The accompanying figures, which are incorporated in and constitute a part of this specification, illustrate several aspects and together with the description serve to explain the principles of the disclosure.

FIG. 1 shows a plot of molar reaction vs. temperature in accordance with an aspect of the present disclosure.

FIG. 2 shows a plot of molar reaction vs. temperature in accordance with an aspect of the present disclosure.

FIG. 3 shows a plot of gas exposure probability vs. temperature in accordance with an aspect of the present disclosure.

FIG. 4 shows a schematic representation of a system in accordance with an aspect of the present disclosure.

FIG. 5 shows a schematic representation of a system in accordance with an aspect of the present disclosure.

FIG. 6 shows a schematic representation of a system in accordance with an aspect of the present disclosure.

FIG. 7 shows a schematic representation of a system in accordance with an aspect of the present disclosure.

FIG. 8 shows a schematic representation of a system in accordance with an aspect of the present disclosure.

FIGS. 9A, 9B, and 9C show comparative schematic representations of systems in accordance with various aspects of the present disclosure.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims.

### DETAILED DESCRIPTION

Methods and apparatus for converting hydrocarbon components in methane feed streams are generally disclosed. As used herein, the term “methane feed stream” includes any feed stream comprising methane. The methane feed streams provided for processing in the reactor (e.g., furnace) generally include methane and form at least a portion of a process stream. The systems and methods presented herein convert at least a portion of the methane to a desired product hydrocarbon compound to produce a product stream having a higher concentration of the product hydrocarbon compound relative to the feed stream.

The term “hydrocarbon stream” as used herein refers to one or more streams that provide at least a portion of the methane feed stream entering the reactor as described herein or are produced from the reactor from the methane feed stream, regardless of whether further treatment or processing is conducted on such hydrocarbon stream. The “hydrocarbon stream” may include the methane feed stream, a reactor effluent stream, a desired product stream exiting a downstream hydrocarbon conversion process, or any intermediate or by-product streams formed during the processes described herein. The hydrocarbon stream may be carried via a process stream line, which includes lines for carrying each of the portions of the process stream described above. The term “process stream” as used herein includes the “hydrocarbon stream” as described above, as well as it may include, alone or in combination, a carrier fluid stream, a fuel stream, an oxygen source stream, or any streams used in the systems and the processes described herein. The process stream may be carried via a process stream line, which includes lines for carrying each of the portions of the process stream described above.

In certain aspects, natural gas (e.g., having greater than 85% methane) may be used as a feedstock to produce ethylene. However, using natural gas in a thermal pyrolysis process may benefit from thermal exposure in a narrow temperature range to maximize yield of acetylene and ethylene, for example. In addition, reduction of the overall energy losses may include minimizing residence time to ensure high efficiencies on a total fuel basis. The systems and methods of the present disclosure provide a burner/combustor/reactor configuration that facilitates control over the pyrolysis conditions to efficiently convert natural gas (and other feeds) from plants to high value acetylene/ethylene and other high value chemicals. Such control also facilitates at least the following: uniform temperature zone for high conversion of methane to acetylene/ethylene; directing the combustion away from the walls to ensure cooler conditions on the wall to ensure long-term durability; operating the combustion zone at temperatures ideal for pyrolysis and minimize heat losses associated with higher temperatures; controlling soot formation to ensure continuous operation of the plant; and removal of a throat section that is the usual hotspot for materials failure. Moreover, the control over the pyrolysis conditions enable online monitoring and steering to offset any feedstock variations or other operating parameters.

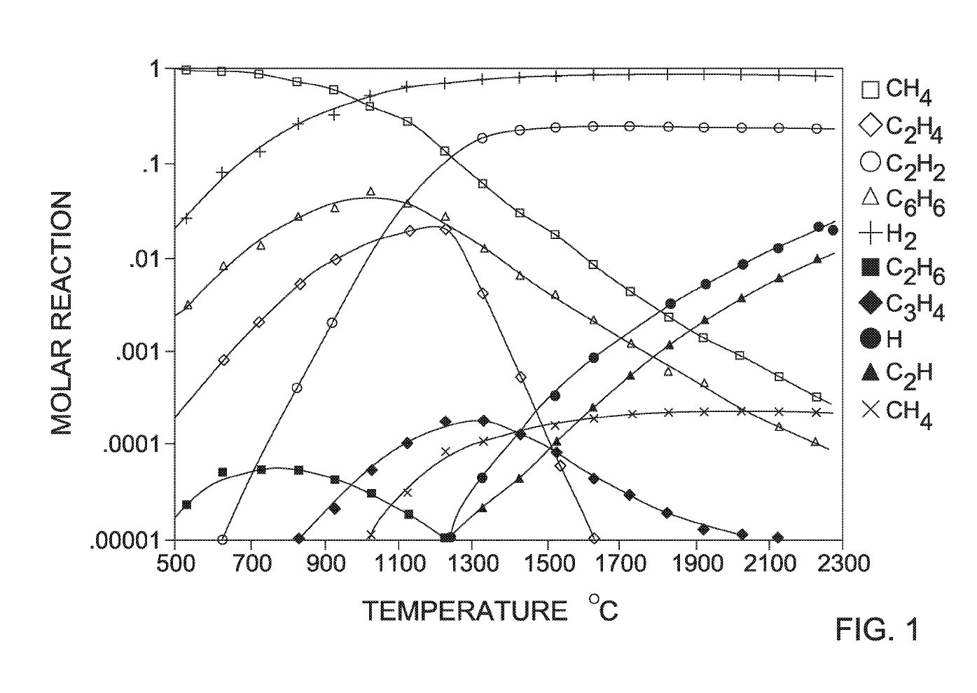

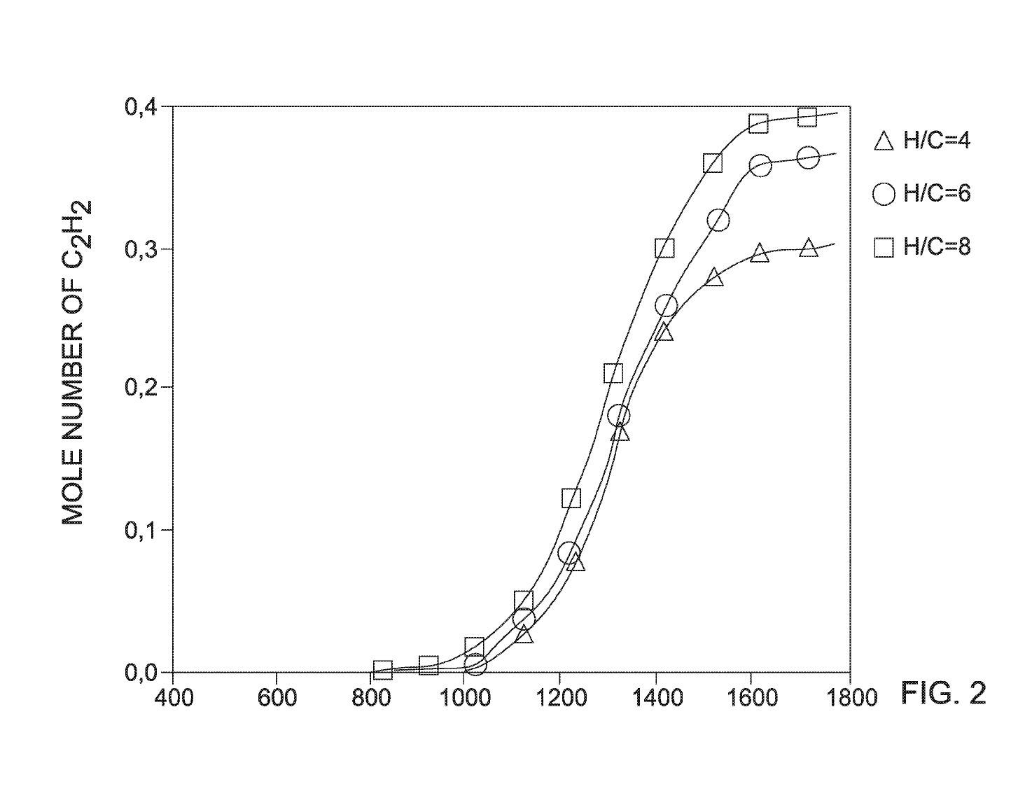

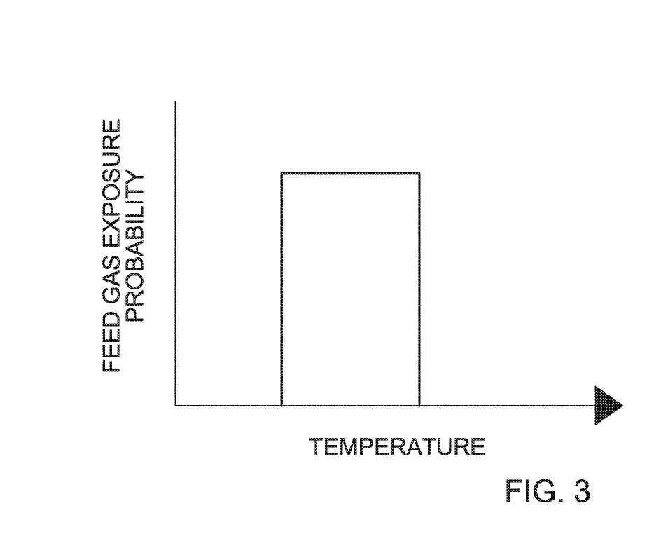

As described herein, the processing of a hydrocarbon stream may be implemented within a desired temperature range. The thermodynamics of the underlying process may be represented by the molar reaction vs. temperature plots illustrated in FIGS. 1-3. As illustrated, the plots indicate that high selectivity and conversion may be exhibited when the pyrolysis reactor is operated over a narrow temperature range. For example, FIG. 1 illustrates illustrates a target temperature range between about 1350° C. and about 1800° C. (shaded) where the ratio of hydrogen to carbon (H/C) is 4 and pressure is 1 atmosphere (atm). As another example, FIG. 2 illustrates a target temperature range between about 1500° C. and about 1800° C. (shaded). The target temperature range may be defined by a metric referenced as gas exposure probability and that may be narrow, as shown in FIG. 3. In accordance with FIGS. 1-3, the gas exposure probability can be defined as the probability of the feedstock gas molecules exposure to a certain temperature range before the molecules are cracked. For example, by supplying volumetric heat to offset the endothermic reactions, a delta function may be created at the point of operating temperature. The systems and methods of the present disclosure facilitate heat transfer to achieve uniform temperature over a narrow range by co-generating heat through the combustion flame alongside pyrolysis reactions.

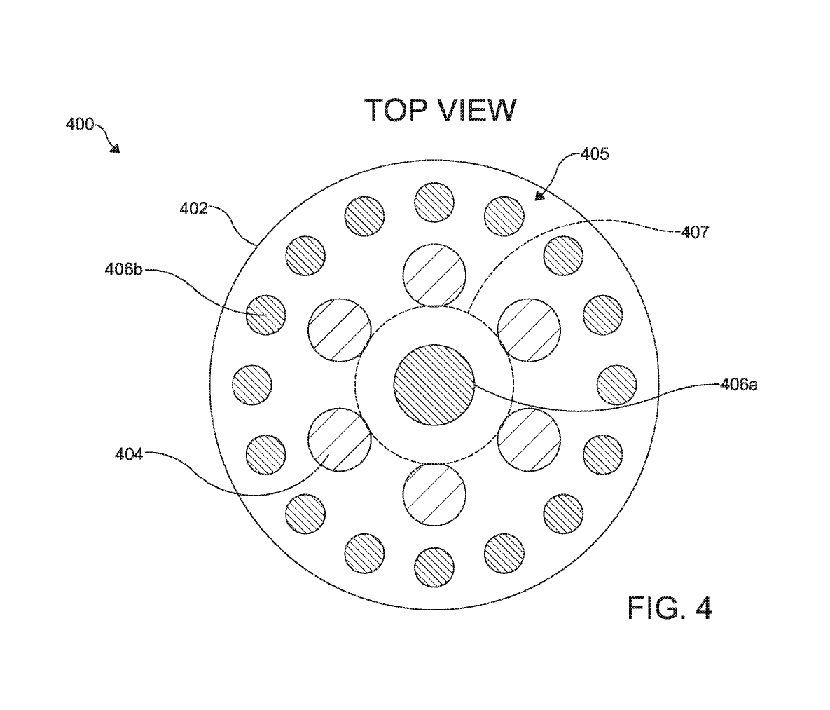

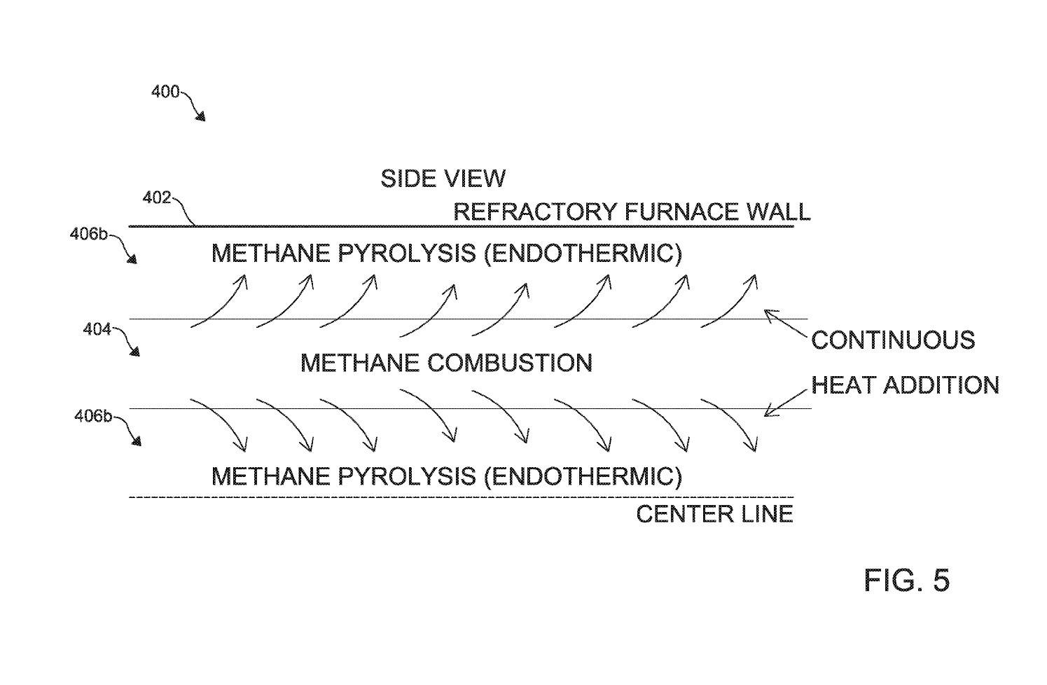

In order to achieve the target temperature range, a concurrent heat generation may be implemented along with pyrolysis, since the pyrolysis reactions are endothermic. In order to achieve the same, the present disclosure provides systems including a burner and feed injector configuration, for example, as shown in FIGS. 4-5. The configuration illustrated in FIGS. 4-5 may facilitate continuous heat addition to maintain a constant temperature within the endothermic pyrolysis zone. As the heat is generated where it is immediately consumed by pyrolysis, the reactor chamber may be operated at temperatures lower than conventional reactors. In addition, the cooler methane gas and the endothermic reactions closer to the walls of the chamber may facilitate lower temperatures at or near the walls, thereby increasing the lifetime of the reactor. The systems and methods of the present disclosure are described in further detail below.

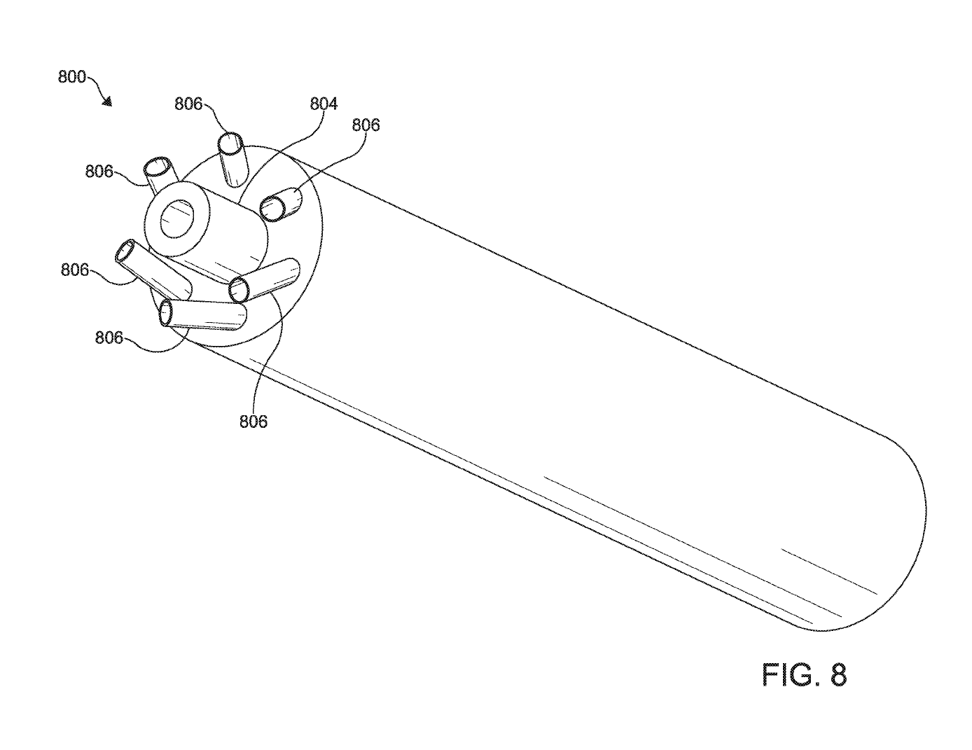

As described herein, in certain aspects, management of the fluid and thermal dynamics inside a reaction chamber may result in increased yield of C2 hydrocarbons (acetylene C₂H₂, ethylene C₂H₄) and hydrogen gas H₂. One mechanism for controlling the fluid dynamics is a swirl introduced by the orientation of one or more injectors, as shown in FIG. 8, and described in further detail below. As an example, the introduction of swirl creates centrifugal force pushing cooler fluid flow (e.g., feedstock) towards the chamber wall or the symmetry plane with the neighboring unit cell, while facilitating the addition of heat through radiation.

### Systems

FIGS. 4-5 illustrate a configuration of a reactor 400, such as a furnace used in processing a hydrocarbon stream. As shown, the reactor 400 may include a wall 402, which may be configured as a refractory furnace wall. The wall 402 may comprise various materials such as stainless steel. The wall 402 may include a cooling jacket (not shown) such as a water cooling system. Other thermal management systems and techniques may be used to control a temperature at or near a surface of the wall 402. For example, the configuration of the reactor 400 may result in a minimization of energy loss and therefore a reduction in temperature at or near the wall 402.

The reactor 400 may include one or more burners 404 configured to introduce heat into a chamber 405 defined by the wall 402. The burners 404 may be supplied with fuel and/or oxygen, which may be combusted to generate heat. As an example, one or more of the burners 404 may be configured as oxy-fuel combustors. Such oxy-fuel combustors may be used for chemical production processes such as hydrocarbon cracking, which breaks the bonds of longer carbon chains resulting in molecularly simpler output chemicals. The combustion undertaken in such processes generates substantial heat and pressure.

The management of heat and pressure must also be conducted according to particular production recipes, which can require consistent heat throughout the chamber 405 and/or particular gradients of heat throughout. Fluid and thermal parameters for such recipes can be controlled, at least in part, based on combustion chamber 405 geometry and the location, orientation, and strength (e.g., firing rate, pressure, fluid velocity) of injectors (inlets) for fuel or other materials into the reactor (e.g., combustion chamber). To fuel the combustion, fluid (gas or liquid) fuel and/or oxygen can be provided through inlets feeding the burners 404. These fluids are ignited within the chamber 405 and combust providing heat and pressure, and in some cases products or byproducts related to the chemical reactions on which chamber output depends, after being provided through inlets about the chamber interior.

The reactor may include one or more injectors 406 configured to introduce fluids into the chamber 405. As an example, the injectors 406 may be configured to introduce a hydrocarbon stream into the chamber 405. As a further example, the injectors may be configured to introduce a methane feed stream into the chamber 405. The injectors 406 may be configured relative to the burners 404, the wall 402, and other injectors 406.

As shown in FIG. 4, a configuration of the burners 404 and injectors 406 may be provided to facilitate continuous heat addition to maintain a constant temperature within the endothermic pyrolysis zone. A plurality of the burners 404 may be arranged in an annular configuration. The annular configuration of the burners 404 may be spaced from the wall 402 and may define an inner volume 407 defined as the space disposed radially inwardly relative to the annular configuration of the burners 404. At least one first injector 406a may be disposed within the inner volume 407 and may be configured to provide a hydrocarbon stream such as a methane feed stream (e.g., natural gas). At least one first injector 406a disposed within the inner volume 407 may be centrally disposed. However, other configurations may be used. In certain aspects, one or more of the burners 404 may be slotted burners to provide enhanced control over flame length, for example.

A plurality of second injectors 406b may be arranged in a configuration and disposed radially outwardly relative to the configuration of the burners 404. The arrangement of the second injectors 406b may be annular or polygonal, or other configurations. The arrangement of the second injectors 406b may be concentrically disposed radially outwardly relative to the configuration of the burners 404. However, other relative arrangements may be used to manage the introduction of a feedstock relative to the burners 404.

As an example, the volume of fluid passing through the at least one first injector 406a may be greater than a volume of a single one of the second injectors 406b. Other configurations of relative flow and volume may be used based on the size of the chamber 405 and the number and size of the burners 404.

As shown in FIG. 5, the combustion of fuel such as methane, for example, by the burners 404 provides continuous heat addition to methane pyrolysis in the presence of a methane feedstock. The methane feedstock may be introduced adjacent the heat source provided by the burners 404. For example, the injectors 406a, 406b may be positioned to introduce methane feed streams adjacent the flame of one or more of the burners 404. Such a configuration may facilitate continuous heat addition to maintain a constant temperature within the endothermic methane pyrolysis zone. As the heat is generated where it is immediately consumed by pyrolysis, the reactor chamber may be operated at temperatures lower than conventional reactors. In addition, the cooler methane gas and the endothermic reactions closer to the walls of the chamber may facilitate lower temperatures at or near the walls, thereby increasing the lifetime of the reactor.

The configuration of the reactor 400 facilitates control features for efficient utilization of the reactor 400 under varying feed or the output conditions. The control features may include a control of the stoichiometry of the burners 404. For example, the burners 404 may be configured to be fuel rich (fuel to oxygen ratio greater than stoichiometry) so that there is limited or no molecular oxygen present in the core. The control features may include a control of injector 406 operation. For example, one or more injectors 406 may be operated in a periodic or pulsed manner. As a further example, pulsing the central injector 406a may provide control over the mixing of the hot combustion air. As yet another example, selective pulsed control of multiple injectors 406 may be used to initiate swirling by out of phase pulsing of the injectors 406. The control features may include a position of one or more of the burners 404 and/or the injectors 406 relative to an orthogonal plane to the wall 403. For example, the burners 404 may be angled radially inwardly to concentrate heat generation, while keeping the wall 402 cooler. As another example, one or more of the injectors 406 may be angled relative to an orthogonal plane to the wall 403, for example, to generate a swirl (see, for example, FIG. 8).

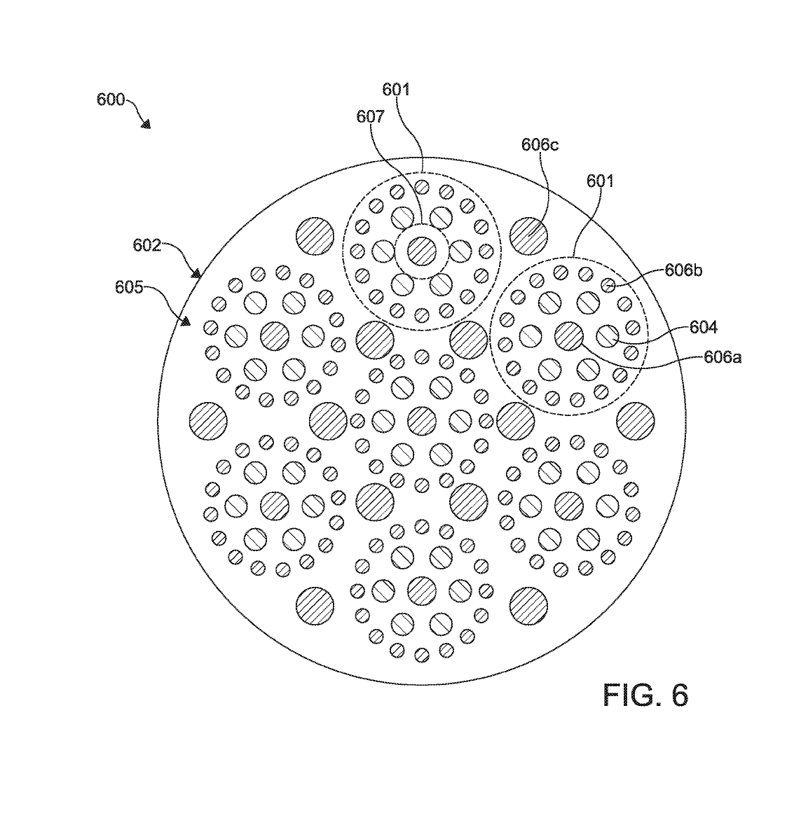

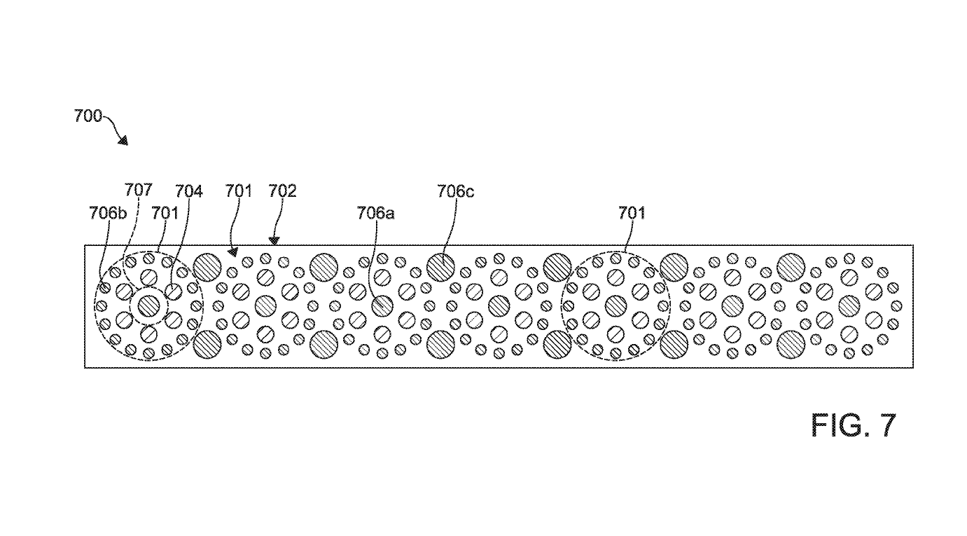

Configuration of reactors such as shown in FIGS. 4-5 may enable scalability. For example, configurations of burners and feedstock injectors may be used in repeated cells, such as pyrolysis cells. FIGS. 6-7 illustrate example reactors 600, 700 including a plurality of pyrolysis cells 601, 701.

FIG. 6 illustrates the reactor 600 comprising a wall 602 defining a chamber 605. A plurality of the pyrolysis cells 601 may be arranged within the chamber 605. The arrangement of the plurality of pyrolysis cells 601 may be generally annular and may surround another one of the pyrolysis cells 601, as shown. Each of the pyrolysis cells 601 may comprises a plurality of burners 604 configured in an arrangement within the pyrolysis cell 601. The arrangement of the plurality of burners 604 of one or more of the pyrolysis cells 601 may be an annular configuration. However, other configurations may be used. Each of the burners may be supplied with a material (e.g., methane, oxygen, etc.) and may facilitate combustion of the material. In certain aspects, the arrangement of the burners 604 may define an inner volume 607 of each respective pyrolysis cell 601 disposed radially inwardly relative to the burners 604 of the pyrolysis cell 601. A first injector 606a may be disposed within the inner volume 607 of a given pyrolysis cell 601 and may be configured to introduce a feedstock (e.g., methane) into the pyrolysis cell 601. As such, the burners 604 of the respective pyrolysis cell 601 provide thermal energy to facilitate thermal pyrolysis of the feedstock.

One or more of the pyrolysis cells 601 may further include a plurality of second injectors 606b. The second injectors 606b may be disposed radially outwardly relative to the arrangement of the plurality of burners 604 for the given pyrolysis cell 601. One or more of the second injectors 606b may be configured to introduce a second feedstock (e.g., methane) into the chamber 605, wherein the plurality of burners 604 provide thermal energy to facilitate thermal pyrolysis of the second feedstock. The second injectors 606b of one or more of the pyrolysis cells 601 may be arranged in an annular or polygonal configuration.

Alternatively or additionally, injectors 606c may be disposed between a first pyrolysis cell and a second pyrolysis cell of the plurality of pyrolysis cells 601. One or more of the injectors 606c may be configured to introduce a feedstock (e.g., methane) into the chamber 605, wherein the plurality of burners 604 provide thermal energy to facilitate thermal pyrolysis of the feedstock.

FIG. 7 illustrates the reactor 700 comprising a wall 702 defining a chamber 705. A plurality of the pyrolysis cells 701 may be arranged within the chamber 705. The arrangement of the plurality of pyrolysis cells 701 may be generally linear, as shown. Each of the pyrolysis cells 701 may comprises a plurality of burners 704 configured in an arrangement within the pyrolysis cell 701. The arrangement of the plurality of burners 704 of one or more of the pyrolysis cells 701 may be an annular configuration. However, other configurations may be used. Each of the burners may be supplied with a material (e.g., methane, oxygen, etc.) and may facilitate combustion of the material. In certain aspects, the arrangement of the burners 704 may define an inner volume 707 of each respective pyrolysis cell 701 disposed radially inwardly relative to the burners 704 of the pyrolysis cell 701. A first injector 706a may be disposed within the inner volume 707 of a given pyrolysis cell 701 and may be configured to introduce a feedstock (e.g., methane) into the pyrolysis cell 701. As such, the burners 704 of the respective pyrolysis cell 701 provide thermal energy to facilitate thermal pyrolysis of the feedstock.

One or more of the pyrolysis cells 701 may further include a plurality of second injectors 706b. The second injectors 706b may be disposed radially outwardly relative to the arrangement of the plurality of burners 704 for the given pyrolysis cell 701. One or more of the second injectors 706b may be configured to introduce a second feedstock (e.g., methane) into the chamber 705, wherein the plurality of burners 704 provide thermal energy to facilitate thermal pyrolysis of the second feedstock. The second injectors 706b of one or more of the pyrolysis cells 701 may be arranged in an annular or polygonal configuration.

Alternatively or additionally, injectors 706c may be disposed between a first pyrolysis cell and a second pyrolysis cell of the plurality of pyrolysis cells 701. One or more of the injectors 706c may be configured to introduce a feedstock (e.g., methane) into the chamber 705, wherein the plurality of burners 704 provide thermal energy to facilitate thermal pyrolysis of the feedstock.

Other configurations of the pyrolysis cells 601, 701 may be used to scale various reactors for various processes such as pyrolysis. In an aspect, acetylene is produced via pyrolysis of natural gas by contacting exhaust gases produced in a combustion chamber. Gases can include natural gas, methane, and/or paraffinic hydrocarbons such as ethane, propane, butane, and/or hexane, alone or in mixed combinations. In alternative or complementary aspects, olefinic hydrocarbons such as ethene, propene, butene, pentene, and/or hexene can be used, alone or in combination with other gases described. In alternative or complementary aspects, alcohols such as methanol, ethanol, propanol, utenol, pentanol, hexanol, and/or amyl alcohol can be used, alone or in combination with other gases described. In aspects, all of the above can be used in varying combinations.

FIG. 8 illustrates an example configuration of a burner 804 and injectors 806 according to aspects of the present disclosure. As shown, a plurality of the injectors 806 may be arranged in an annular configuration. The annular configuration of the injectors 806 may be spaced from the burner 804 disposed radially inward from the configuration of injectors 806. As an example, the burner 804 may be centrally disposed within the configuration of the injectors 806. Other configurations and numbers of burners 804 and injectors 806 may be used. Similar configurations may also be used for an arrangement of injectors 806, where a central injector 806 replaces the burner 804.

The injectors 806 may be configured at an angle relative to an orthogonal axis. For example, each of the injectors 806 configured in the annular configuration may be angled at 30 degrees from the orthogonal axis in order to create a swirl effect via injected fluid. Such a swirl effect may create fluid forces to control a movement of fluids and products within a chamber such as the various chambers described herein. Any number of the injectors 806 having various angles and sizes may be used based on the chamber size, reaction, and desired production.

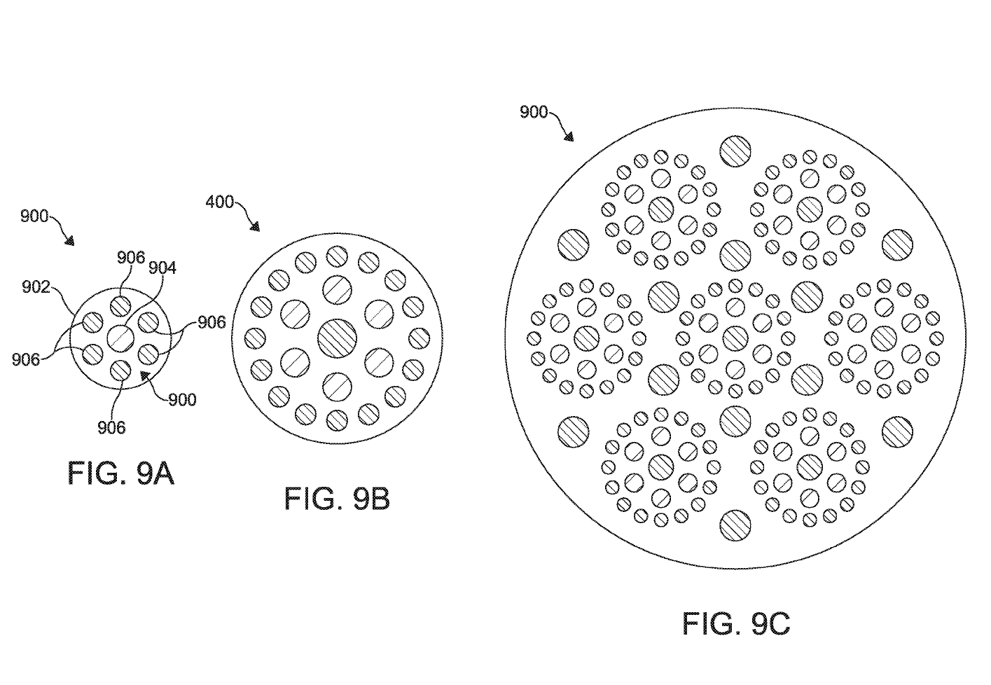

FIGS. 9A-9C illustrate an illustrative comparison of various scaled reactors. FIG. 9A illustrates an example configuration of a reactor 900 comprising a wall 902 defining a chamber 905. As shown, a plurality of the injectors 906 may be arranged in an annular configuration. The annular configuration of the injectors 906 may be spaced from a burner 904 disposed radially inward from the configuration of injectors 906. As an example, the burner 904 may be centrally disposed within the configuration of the injectors 906. Such a configuration may produce a pyrolysis zone, as described herein. As an example, the reactor may be representative of a production scale of 20 million (or thousand thousand) British Thermal units per hour (MMBTU/hr). Other configurations and numbers of burners 904 and injectors 906 may be used to scale an overall production. FIG. 9B illustrates the reactor 400 (FIG. 4), the configuration of which may be representative of a production scale of 120 MMBTU/hr. For additional comparison, FIG. 9C illustrates the reactor 600 (FIG. 6), the configuration of which may be representative of a production scale of 840 MMBTU/hr. Although the production rates are used as examples, it is understood that the arraignments described herein provide flexibility in scaling various reactors to custom production rates, while managing the issues of the conventional systems described herein.

### METHOD

Various processes may make use of the reactors described herein. As an example, a method of processing a hydrocarbon feed may comprise causing combustion of a fuel (e.g., methane) to generate heat within a chamber via a plurality of burners configured in an arrangement within the chamber. The arrangement may define an inner volume disposed radially inwardly relative thereto. The method may also comprise causing a feedstock (e.g., methane) to be introduced in the chamber via an injector disposed within the inner volume. As such, the heat generated by the combustion of the fuel facilitates thermal pyrolysis of the feedstock. The method may further comprise causing feedstock to be introduced in the chamber via a second injector disposed radially outwardly relative to the arrangement of the plurality of burners. Such arrangements may facilitate continuous heat addition to maintain a constant temperature within an endothermic pyrolysis zone. As the heat is generated where it is immediately consumed by pyrolysis, the reactor chamber may be operated at temperatures lower than conventional reactors. In addition, the cooler methane gas and the endothermic reactions closer to the walls of the chamber may facilitate lower temperatures at or near the walls, thereby increasing the lifetime of the reactor.

While aspects of the present disclosure can be described and claimed in a particular statutory class, such as the system statutory class, this is for convenience only and one of skill in the art will understand that each aspect of the present disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### ASPECTS

The disclosed systems and methods include at least the following aspects.

Aspect 1: A system comprising: a wall defining a chamber; a plurality of burners configured in an arrangement within the chamber, wherein each of the burners is supplied with a material and facilitates combustion of the material, and wherein the arrangement defines an inner volume disposed radially inwardly relative thereto; and an injector disposed within the inner volume and configured to introduce a feedstock into the chamber, wherein the plurality of burners provide thermal energy to facilitate thermal pyrolysis of the feedstock.

Aspect 2: A system consisting essentially of: a wall defining a chamber; a plurality of burners configured in an arrangement within the chamber, wherein each of the burners is supplied with a material and facilitates combustion of the material, and wherein the arrangement defines an inner volume disposed radially inwardly relative thereto; and an injector disposed within the inner volume and configured to introduce a feedstock into the chamber, wherein the plurality of burners provide thermal energy to facilitate thermal pyrolysis of the feedstock.

Aspect 3: A system consisting of: a wall defining a chamber; a plurality of burners configured in an arrangement within the chamber, wherein each of the burners is supplied with a material and facilitates combustion of the material, and wherein the arrangement defines an inner volume disposed radially inwardly relative thereto; and an injector disposed within the inner volume and configured to introduce a feedstock into the chamber, wherein the plurality of burners provide thermal energy to facilitate thermal pyrolysis of the feedstock.

Aspect 4. The system of any one of aspects 1-3, wherein the arrangement of the plurality of burners is dependent on a cross-sectional shape of the chamber.

Aspect 5. The system of any one of aspects 1-4, wherein the arrangement of the plurality of burners comprises an annular configuration.

Aspect 6. The system of any one of aspects 1-5, wherein the material comprises methane, or oxygen, or a combination thereof

Aspect 7. The system of any one of aspects 1-6, wherein the feedstock comprises a hydrocarbon.

Aspect 8. The system of any one of aspects 1-7, further comprising a plurality of second injectors disposed radially outwardly relative to the arrangement of the plurality of burners, wherein each of the second injectors is configured to introduce a second feedstock into the chamber, wherein the plurality of burners provide thermal energy to facilitate thermal pyrolysis of the second feedstock.

Aspect 9. The system of aspect 8, wherein the plurality of second injectors is arranged in an annular or polygonal configuration.

Aspect 10. The system of aspect 8, wherein the plurality of second injectors is arranged in an annular configuration.

Aspect 11. The system of aspect 8, wherein the plurality of second injectors is arranged in a polygonal configuration.

Aspect 12. The system of any one of aspects 8-11, wherein the second feedstock comprises methane.

Aspect 13. A system comprising: a wall defining a chamber; and a plurality of pyrolysis cells arranged within the chamber, wherein each pyrolysis cell comprises: a plurality of burners configured in an arrangement within the pyrolysis cell, wherein each of the burners is supplied with a material and facilitates combustion of the material, and wherein the arrangement defines an inner volume disposed radially inwardly relative thereto; and an injector disposed within the inner volume and configured to introduce a feedstock into the pyrolysis cell, wherein the plurality of burners provide thermal energy to facilitate thermal pyrolysis of the feedstock.

Aspect 14. The system of aspect 13, wherein the arrangement of the plurality of pyrolysis cells is linear or annular.

Aspect 15. The system of any one of aspects 13-14, wherein the arrangement of the plurality of burners in each pyrolysis cell comprises an annular configuration.

Aspect 16. The system of any one of aspects 13-15, wherein the material comprises methane, or oxygen, or a combination of both.

Aspect 17. The system of any one of aspects 13-16, wherein the feedstock comprises a hydrocarbon.

Aspect 18. The system of any one of aspects 13-17, wherein at least one of the pyrolysis cells further comprises a plurality of second injectors disposed radially outwardly relative to the arrangement of the plurality of burners, wherein each of the second injectors is configured to introduce a second feedstock into the chamber, wherein the plurality of burners provide thermal energy to facilitate thermal pyrolysis of the second feedstock.

Aspect 19. The system of aspect 18, wherein the plurality of second injectors is arranged in an annular or polygonal configuration.

Aspect 20. The system of any one of aspects 13-19, further comprising one or more second injectors disposed between a first pyrolysis cell and a second pyrolysis cell of the plurality of pyrolysis cells, wherein each of the second injectors is configured to introduce a second feedstock into the chamber, wherein the plurality of burners provide thermal energy to facilitate thermal pyrolysis of the second feedstock.

Aspect 21. The system of any one of aspects 13-20, wherein the second feedstock comprises methane.

Aspect 22. A method of processing a hydrocarbon stream, the method comprising: causing combustion of a fuel to generate heat within a chamber via a plurality of burners configured in an arrangement within the chamber, wherein the arrangement defines an inner volume disposed radially inwardly relative thereto; and causing a feedstock to be introduced in the chamber via an injector disposed within the inner volume, wherein the heat generated by the combustion of the fuel facilitates thermal pyrolysis of the feedstock.

Aspect 23. A method of processing a hydrocarbon stream, the method consisting essentially of: causing combustion of a fuel to generate heat within a chamber via a plurality of burners configured in an arrangement within the chamber, wherein the arrangement defines an inner volume disposed radially inwardly relative thereto; and causing a feedstock to be introduced in the chamber via an injector disposed within the inner volume, wherein the heat generated by the combustion of the fuel facilitates thermal pyrolysis of the feedstock.

Aspect 24. A method of processing a hydrocarbon stream, the method consisting of: causing combustion of a fuel to generate heat within a chamber via a plurality of burners configured in an arrangement within the chamber, wherein the arrangement defines an inner volume disposed radially inwardly relative thereto; and causing a feedstock to be introduced in the chamber via an injector disposed within the inner volume, wherein the heat generated by the combustion of the fuel facilitates thermal pyrolysis of the feedstock.

Aspect 25. The method of any one of aspects 22-24, further comprising causing feedstock to be introduced in the chamber via a second injector disposed radially outwardly relative to the arrangement of the plurality of burners.

Aspect 26. The method of any one of aspects 22-25, wherein one or more of the fuel and the feedstock comprise methane.

It is to be understood that the terminology used herein is for the purpose of describing particular aspects only and is not intended to be limiting. Although any methods and materials similar or equivalent to those described herein can be used in the practice or testing of the present disclosure, example methods and materials are now described. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present disclosure is not entitled to antedate such publication by virtue of prior disclosure. Further, the dates of publication provided herein may be different from the actual publication dates, which can require independent confirmation.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, example methods and materials are now described.

As used in the specification and the appended claims, the singular forms “a,” “an” and “the” include plural referents unless the context clearly dictates otherwise. Thus, for example, reference to “an injector” may include one or more injectors.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not. For example, the phrase “optionally substituted alkyl” means that the alkyl group can or cannot be substituted and that the description includes both substituted and un-substituted alkyl groups.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

Each of the materials disclosed herein are either commercially available and/or the methods for the production thereof are known to those of skill in the art.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A system comprising:
a wall defining a chamber;
a plurality of burners configured in an arrangement within the chamber, wherein each of the burners is supplied with a material and facilitates combustion of the material, and wherein the arrangement defines an inner volume disposed radially inwardly relative thereto; and
an injector disposed within the inner volume and configured to introduce a feedstock into the chamber, wherein the plurality of burners provide thermal energy to facilitate thermal pyrolysis of the feedstock.

2. The system of claim 1, wherein the arrangement of the plurality of burners is dependent on a cross-sectional shape of the chamber.

3. The system of claim 1, wherein the arrangement of the plurality of burners comprises an annular configuration.

4. The system of claim 1, wherein the material comprises methane, oxygen, or a combination thereof

5. The system of claim 1, wherein the feedstock comprises a hydrocarbon.

6. The system of claim 1, further comprising a plurality of second injectors disposed radially outwardly relative to the arrangement of the plurality of burners, wherein each of the second injectors is configured to introduce a second feedstock into the chamber, wherein the plurality of burners provide thermal energy to facilitate thermal pyrolysis of the second feedstock.

7. The system of claim 6, wherein the plurality of second injectors is arranged in an annular or polygonal configuration.

8. The system of claim 6, wherein the second feedstock comprises methane.

9. A system comprising:
a wall defining a chamber; and
a plurality of pyrolysis cells arranged within the chamber, wherein each pyrolysis cell comprises:
a plurality of burners configured in an arrangement within the pyrolysis cell, wherein each of the burners is supplied with a material and facilitates combustion of the material, and wherein the arrangement defines an inner volume disposed radially inwardly relative thereto; and
an injector disposed within the inner volume and configured to introduce a feedstock into the pyrolysis cell, wherein the plurality of burners provide thermal energy to facilitate thermal pyrolysis of the feedstock.

10. The system of claim 9, wherein the arrangement of the plurality of pyrolysis cells is linear or annular.

11. The system of claim 9, wherein the arrangement of the plurality of burners in each pyrolysis cell comprises an annular configuration.

12. The system of claim 9, wherein the material comprises methane, oxygen, or a combination thereof.

13. The system of claim 9, wherein the feedstock comprises a hydrocarbon.

14. The system of claim 9, wherein at least one of the pyrolysis cells further comprises a plurality of second injectors disposed radially outwardly relative to the arrangement of the plurality of burners, wherein each of the second injectors is configured to introduce a second feedstock into the chamber, wherein the plurality of burners provide thermal energy to facilitate thermal pyrolysis of the second feedstock.

15. The system of claim 14, wherein the plurality of second injectors is arranged in an annular or polygonal configuration.

16. The system of claim 9, further comprising one or more second injectors disposed between a first pyrolysis cell and a second pyrolysis cell of the plurality of pyrolysis cells, wherein each of the second injectors is configured to introduce a second feedstock into the chamber, wherein the plurality of burners provide thermal energy to facilitate thermal pyrolysis of the second feedstock.

17. The system of claim 14, wherein the second feedstock comprises methane.

18. A method of processing a hydrocarbon stream, the method comprising:
causing combustion of a fuel to generate heat within a chamber via a plurality of burners configured in an arrangement within the chamber, wherein the arrangement defines an inner volume disposed radially inwardly relative thereto; and
causing a feedstock to be introduced in the chamber via an injector disposed within the inner volume, wherein the heat generated by the combustion of the fuel facilitates thermal pyrolysis of the feedstock.

19. The method of claim 18, further comprising causing feedstock to be introduced in the chamber via a second injector disposed radially outwardly relative to the arrangement of the plurality of burners.

20. The method of claim 18, wherein one or more of the fuel and the feedstock comprise methane.
